(19) United States
(12) Patent Application Publication
(10) Pub. No.: US 2024/0261319 A1
(43) Pub. Date: Aug. 8, 2024

(54) ANTI-FUNGAL COMPOSITION DERIVED FROM HESPERALOE

(71) Applicant: Kimberly-Clark Worldwide, Inc., Neenah, WI (US)

(72) Inventors: Ning Wei, Roswell, GA (US); Thomas G. Shannon, Neenah, WI (US)

(21) Appl. No.: 18/571,905

(22) PCT Filed: Jun. 21, 2021
(86) PCT No.: PCT/US21/38279

§ 371 Date: Dec. 19, 2023

(51) Int. Cl. A61K 31/715; A01N 43/90; A01P 3/00; A61K 36/88; A61P 31/10; C07J 71/00

(52) U.S. Cl. CPC A61K 31/715; A01N 43/90; A01P 3/00; A61K 36/88; A61P 31/10; C07J 71/0005; A61K 2236/15; A61K 2236/39

(57) ABSTRACT

Disclosed are anti-fungal compositions prepared from non-woody plants of the genus Hesperaloe and processes for preparing the same. The anti-fungal compositions preferably comprise at least one saponin. In certain instances, the process includes providing biomass derived from non-woody plants of the genus Hesperaloe, milling the biomass, washing the biomass with a solvent to yield a crude extract and further purifying the crude extract by filtration to remove water insoluble compositions such as fibers, fines, epidermal debris and lipids. The compositions are particularly useful in preventing or inhibiting the growth of fungal organisms, particularly those in the Candida genus.

### BACKGROUND

Plants produce a vast and diverse assortment of organic compounds, the great majority of which do not appear to participate directly in their growth and development. These substances, traditionally referred to as secondary metabolites or plant natural products, often are distributed among limited taxonomic groups within the plant kingdom. The functions of secondary metabolites remain largely unknown, although a number of compounds have been associated with attributes useful to the plants e.g. protection against herbivores and protection against microbial infection, as attractants for pollinators and seed-dispersing animals, and as compounds that influence competition among plant species (allelochemicals). There is a growing interest in plant natural products, since these products often have a wide range of applications in different kinds of industries, including pharmaceutical industries, cosmetic industries, food industries, detergent industries, and the like.

A particular group of plant secondary metabolites of interest are saponins. Saponins are glycosylated compounds classified as either triterpenoids, steroids, or steroidal glycoalkaloids. Saponins consist of one or two sugar moieties which are coupled to the aglycon (mono- and bisdesmosides, respectively). Saponins can be hydrolyzed to sapogenins and sugar moieties by acid hydrolysis or enzymatic methods. Saponins are generally water soluble high molecular weight compounds with molecular weights ranging from 600 to more than 2,000 daltons.

The asymmetric distribution of their hydrophobic (aglycone) and hydrophilic (sugar) moieties confers an amphipathic character to these compounds which are largely responsible for their detergent-like properties. The ability of lowering surface tension makes saponins potentially well suited for use in the cosmetic and in the detergent industries.

Saponins also have the ability of forming insoluble complexes with cholesterol, which makes some of them suitable for use in the pharmaceutical industry as cholesterol lowering agents. Other saponins are associated with formation of immunostimulating complexes that are useful in vaccine strategies.

Currently, a major limitation to the broad exploitation of saponins is the fact that commercially available saponins are relatively expensive. The expenses is due in large part to the limited number of plant extracts having significant amounts of saponins. Currently, commercially available plant extracts containing saponins include Saponaria officinalis, Quillaia bark and stem, Castanea sativa seeds, and extracts of various Yucca species.

Plant extracts containing saponins are thus of general interest within a wide range of different industries. There is therefore a growing need in the art for alternative sources of saponin extracts and these plant sources should preferably be cheap, easy to obtain, and preferably the saponin content should be relatively high.

### SUMMARY

The present inventors have now discovered that Hesperaloe biomass, particularly the above ground portion of a Hesperaloe plant and more particularly the portion of the Hesperaloe plant above the crown, may be processed prior to pulping to extract water soluble solids such as inorganic salts, saccharides and saponins. In certain instances, the extraction may comprise milling biomass derived from a non-woody plants of the genus Hesperaloe to yield a crude extract, also referred to herein as a juice, which may be further processed to concentrate or isolate specific water soluble solids. In other instances, the extraction process may comprise milling the biomass and washing the milled biomass with an aqueous solvent to yield a juice, separating water insoluble solids from the juice, and optionally concentrating the juice.

Of particular interest are saponins derived from Hesperaloe biomass, particularly one or more saponins selected from the group consisting of 25(27)-dehydrofucreastatin (FIG. 4A), 5(6),25(27)-disdehydroyuccaloiside C (FIG. 4B), 5(6)-disdehydroyuccaloiside C (FIG. 4C), furcreastatin and yuccaloiside C. Saponins derived from Hesperaloe may be useful in a wide range of applications including, for example, improving crop resistance to a variety fungi and also as a pharmaceutical against human and animal fungal-induced diseases.

Accordingly, in one embodiment the present invention provides a composition comprising one or more saponins selected from the group consisting of 25(27)-dehydrofucreastatin (FIG. 4A), 5(6),25(27)-disdehydroyuccaloiside C (FIG. 4B), 5(6)-disdehydroyuccaloiside C (FIG. 4C), furcreastatin and yuccaloiside C, wherein the composition has activity against a large variety of fungal organisms associated with diseases in plants, animals, and humans.

In other embodiments the present invention comprises a method of preparing an anti-fungal composition from Hesperaloe biomass comprising the steps milling biomass derived from non-woody plants of the genus Hesperaloe including, for example, Hesperaloe funifera, Hesperaloe nocturna, Hesperaloe parviflora, and Hesperaloe chiangii and extracting hydrophilic compounds from the biomass using a suitable solvent. The hydrophilic compounds may be further purified or isolation by extraction or separation to yield one or more saponins. In certain instances, separation may be effected by centrifugation with recovery and filtration of the supernatant. The saponins are eluted from the supernatant with an aqueous methanol or other polar solvent. The saponins may then be further purified from the methanol eluate, such as by liquid chromatography, high performance liquid chromatography (HPLC), or the like.

In other embodiments, the water soluble fraction extracted from Hesperaloe biomass may be concentrated and treated to remove impurities to yield a composition comprising a mixture of saponins having anti-fungal activity. For example, in certain embodiments, the invention provides a process for preparing a composition comprising one or more saponins selected from the group consisting of 25(27)-dehydrofucreastatin (FIG. 4A), 5(6),25(27)-disdehydroyuccaloiside C (FIG. 4B), 5(6)-disdehydroyuccaloiside C (FIG. 4C), furcreastatin and yuccaloiside C. The process comprises the steps of mixing the water soluble fraction with a salt and a solvent to form a first solution, adjusting the pH of the first solution to about 6.0 to about 7.0, adding at least one phosphate to the first solution to form an ion-polysaccharides precipitated and removing the precipitated, such as by filtration, to yield a composition comprising a mixture of saponins that is substantially free from saponins.

In still other embodiments, the present invention provides a substantially pure saponin composition derived from Hesperaloe biomass, wherein the substantially pure saponin composition has anti-fungal activity. In its substantially pure form, the saponin composition may be substantially free from phenolic compounds (such as tannins, quercetin, leucocyanidin, kaempferol, among others), organic acids (such as caffeic acid, gallic acid, coumaric acid), free saccharides, lipids, and nitrogen-containing compounds, such as proteins. The terms free polysaccharide and free phenolic compounds generally refer to compounds that are not part of a saponin. Thus, while saponins may contain one or more saccharides, such saccharides are not “free” saccharides.

### DESCRIPTION OF THE DRAWINGS

FIGS. 1A and 1B illustrate a triterpenoid saponin and a steroidal saponin, respectively;

FIGS. 2A-C illustrate various novel saponins extracted from non-woody plants of the genus Hesperaloe according to the present invention including, 25(27)-dehydrofucreastatin (FIG. 2A), 5(6),25(27)-disdehydroyuccaloiside C (FIG. 2B), and 5(6)-disdehydroyuccaloiside C (FIG. 2C);

FIG. 3 is a schematic of the extraction of Hesperaloe funifera as described in the Example;

FIG. 4 is a schematic of the extraction of CHCl₃—MeOH fraction as described in the Example;

FIG. 5 is a schematic of the extraction of MeOH fraction as described in the Example;

FIG. 6 is a schematic of the extraction of MeOH-water fraction as described in the Example;

FIG. 7 is a schematic of the extraction of hot water fraction as described in the Example; and

FIG. 8 is a graph illustrating the anti-fungal properties of saponins according to certain embodiments of the present invention.

### DEFINITIONS

As used herein the term “biomass” generally refers to whole plants and plant organs (i.e., leaves, stems, flowers, roots, etc.) of the genus Hesperaloe including, for example, Hesperaloe funifera, Hesperaloe nocturna, Hesperaloe parviflora, and Hesperaloe chiangii. In particularly preferred instances water soluble solids may be prepared from biomass consisting essentially of the above ground portion of the Hesperaloe plant and more particularly the portion of the Hesperaloe plant above the crown and still more preferable the leaves of the Hesperaloe plant.

As used herein the term “bagasse” generally refers to biomass that has been subjected to an extraction process such as, for example, continuous solvent extraction or milling, so that the resulting solids have less water soluble solids than the biomass from which it is derived. In certain preferred embodiments bagasse is prepared by subjecting biomass to high pressure, such as by milling. High pressure may be achieved by using compression pressure, such as that provided by machines such one or more opposed counter-rotating rolls, a mechanical press, a screw press as well as by direct hydraulic pressure and other processes to apply pressure to the biomass and remove intercellular and intracellular liquid.

As used herein the term “milling” generally refers to the application of sufficient pressure to force the intercellular and intracellular liquid from the biomass.

As used herein, the term “saccharide” is used interchangeably with the terms “polysaccharide,” “oligosaccharide” and “sugar” the definitions of which are well known to those skilled in the art of carbohydrate chemistry. It should be noted that the saccharides can be in the form of mono-, oligo- and/or polysaccharides. Preferably saccharides are water soluble and do not include cellulose, hemicellulose or mono-, oligo- and/or polysaccharides bound to other compounds, such as glycosides (arabinose, glucose, galactose, xylose, and glucuronic acid) bound to a triterpenoid to form a saponin.

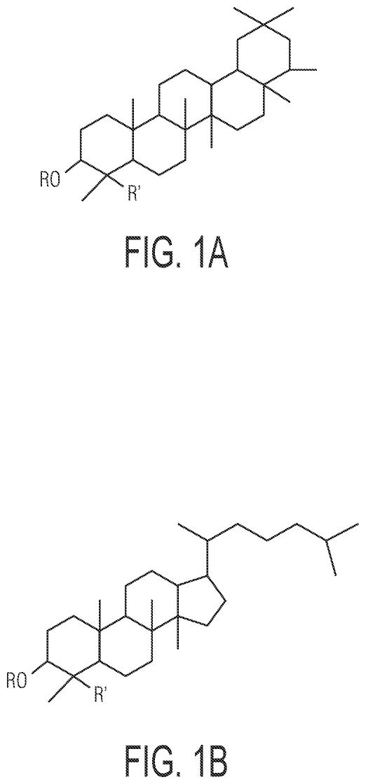

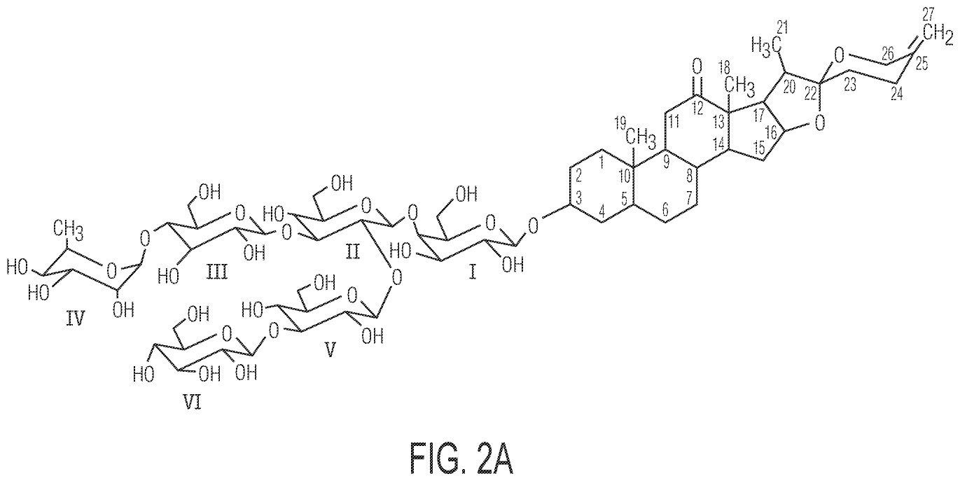

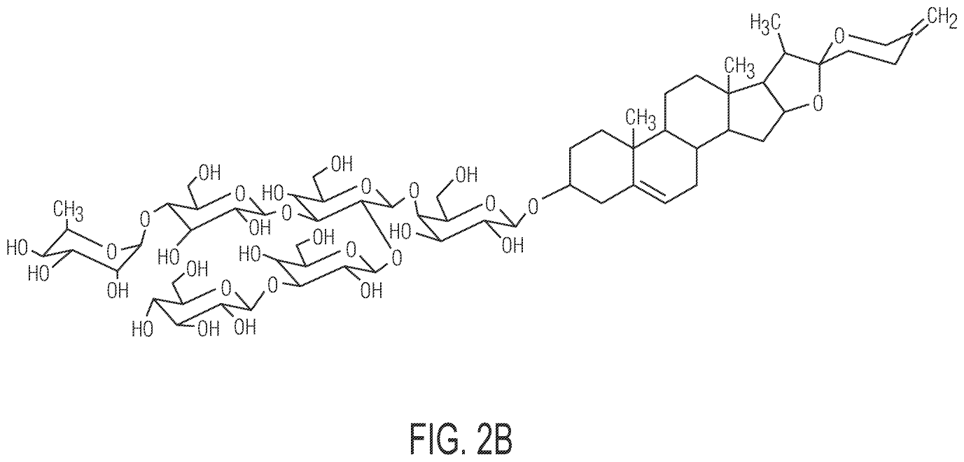

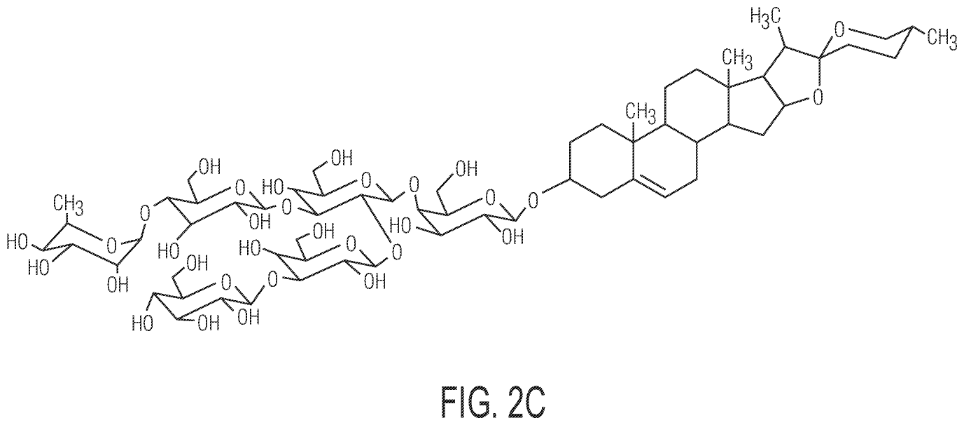

As used herein the term “saponin” generally refers to glycosides comprising a sugar component referred to as a glycone and a non-sugar component referred to as an aglycone. Depending on the structure of the aglycone the saponin may be classified as a triterpenoid saponin, illustrated in FIG. 1A, or to steroidal saponin, illustrated in FIG. 1B. The aglycone portion of the saponin may be either a pentacyclic triterpenoid or a tetracyclic triterpenoid, both of which contain 30 carbon atoms. Whether steroidal or triterpenoid, saponins may be mono, bi- or tridesmodic. Monodesmodic saponins have a single saccharide, normally attached at C-3. Bidesmodic saponins have two saccharides, often with one attached through an ether linkage at C-3 and the other either attached through an ester linkage at C-28 or through an ether linkage at C-20 (pentacyclic and tetracyclic triterpene saponins, respectively), or through an ether linkage at C-26 (furostane saponins). In certain instances, Hesperaloe biomass may comprises at least about 5 wt % of total saponins, such as from about 5 to about 15 wt %, such as from about 8 to about 12 wt %, based upon the bone dry weight of the biomass. Total saponins may be determined as described in the Test Methods section below.

As used herein the term “water soluble solids” generally refers to dry matter which remains after the extract has been centrifuged, filtered and all water is evaporated. The procedure for measuring water soluble solids of a biomass extract of the present invention is described in detail in the Test Methods section below. Water soluble solids may be expressed on a percentage basis relative to the mass of bone dry biomass.

As used herein the term “water insoluble solids” generally refer to the fraction of extract that is removed by centrifugation and filtration in the course of measuring water soluble solids, as described in the Test Methods section below.

### DETAILED DESCRIPTION

This invention relates to saponins derived from non-woody plants and more particularly the non-woody plants of the genus Hesperaloe. In particular, the present invention is directed towards processing biomass derived from non-woody plants of the genus Hesperaloe including, for example, Hesperaloe funifera, Hesperaloe noctuma, Hesperaloe parviflora, and Hesperaloe chiangii. Saponins prepared from non-woody plants of the genus Hesperaloe, particularly Hesperaloe funifera, are useful as anti-fungal agents. The anti-fungal compositions of the present invention may be used in a wide range of applications, for example, improving crop resistance to a variety of fungi and also as a pharmaceutical against human and animal fungal-induced diseases.

Anti-fungal compositions of the present invention may be prepared from non-woody plants of the genus Hesperaloe by extracting biomass, particularly the leaves and more particularly the leaves above the crown of the plant, with at least one solvent selected from the group consisting of water, methanol, ethanol, butanol, and isopropanol and mixtures thereof. For example, in one embodiment, the process comprises contacting biomass with an extractant solution comprising water and separating the water soluble fraction from the insoluble biomass fraction. In other embodiments the extractant solution may comprise, in addition to water, a surfactant, a solvent and optionally extract-bearing juice. The extract-bearing juice can come from, for example, an earlier extraction step or an earlier milling step.

Milling operations useful for separating the bagasse and water soluble solids may include a roll, screw, and other forms of presses. The solvent may be introduced during the milling process to extract hydrophilic solids from the bagasse. In certain instances, the bagasse can then be contacted with the juice in a subsequent milling step, often referred to as imbibing. In certain instances, the biomass may be cut to size and cleaned prior to milling.

A simple water extraction of Hesperaloe biomass may yield a crude aqueous extract comprising saccharides, polysaccharides, inorganic salts, saponins and sapogenins. A crude extract may also be produced using methanol as a solvent, or a mixture of methanol and water, to extract biomass, which may have been previously extracted with acetone or diethyl ether to remove lipids and pigments. In other instances, the biomass may be extracted with a 4:1 ethanol-water solvent, followed by subsequent defatting of the extract with a non-polar solvent such as hexane. In certain instances, the defatted extract may be subjected to further treatment to isolate specific water soluble components, such as saponins, which may be purified from the defatted extract by mixing with butanol and separating the butanol phase to yield a mixture of saponins that are substantially free from proteins and free saccharides and polysaccharides.

Hot aqueous extractants can also be used. For example, in one embodiment water soluble solids may be extracted from Hesperaloe biomass, particularly the leaves, by extracting the biomass with hot aqueous ethanol or isopropanol (75 to 95% by weight alcohol). The aqueous alcohol extraction fluid may then be filtered and concentrated, and the fat-soluble material may be removed by mixing the extraction fluid with a non-polar solvent such as hexane. A substantially pure saponin composition may then be prepared by further extracting defatted extract with a polar solvent such as butanol.

For the purpose of preparing the compositions of the present invention, and for use in the present method, a simple aqueous extract may be preferred, although other extraction methods are within the scope of the present invention. In a particularly preferred embodiment, Hesperaloe biomass may be cut to size, pressed, and extracted with an aqueous solvent to remove water soluble extracts such as inorganic salts, saccharides, polysaccharides, organic acids and saponins. The water soluble extracts are collected and may be concentrated by techniques well known in the art such as, for example, evaporation, spray-drying, drum drying and the like. The extract may be concentrated until it has a solids content of about 20 to about 100% solids by weight, such as from about 20 to about 95% solids by weight, such as from about 20 to about 80% solids by weight.

In certain embodiments water soluble extracts may be concentrated by feeding the extract solution to atomizing equipment. Suitable atomizing equipment includes, but is not limited to, a rotary wheel atomizer, a pressure nozzle atomizer, and a dual fluid nozzle atomizer. Rotary wheel, pressure nozzle and dual fluid nozzle atomizers are known to those of ordinary skill in the art and include those in spray dryers commercially available from a variety of sources, such as GEA Process Engineering.

In other embodiments the water soluble solids may be recovered from biomass by diffusion. In diffusion, the biomass brought into contact with the liquid to extract the liquid components. Usually, the biomass is prepared by first cutting, but not shearing or crushing so as to minimize the damage to fibers and avoid the creation of an excessive amount of fines. The prepared biomass is then washed repeatedly, usually using a solvent, to extract the liquid contained in the biomass. The solvent can be any of the foregoing solvents. An exemplary treatment solvent is water, particularly hot water such as water heated to a temperature from about 40 to about 90° C. The solvent can be circulated and reused so that the solvent used for a first extraction is reused as a solvent to extract subsequent prepared biomass.

Various types of diffusers are known in the art and can be adapted for use with biomass as described herein. Suitable diffusers include a ring diffuser, a tower diffuser, or a drum diffuser. Exemplary diffusion systems are discussed, for example, in U.S. Pat. Nos. 4,182,632, 4,751,060, 5,885,539 and 6,193,805 the contents of which are hereby incorporated in a manner consistent with the present disclosure. Numerous other diffusion methods and devices for the diffusion method are known and can be adapted for use in the methods described herein. One such diffuser is the continuous-loop, counter-current, shallow-bed Crown Model III Percolation Extractor, commercially available from Crown Iron Works, Blaine, MN.

The biomass, cut or uncut, may be extracted by any suitable extraction process as discussed above. In a particularly preferred embodiment, the solvent used for extraction comprises water. One of skill in the art will recognize the ratio of extraction solvent to biomass will vary based on the solvent, the amount of biomass to be extracted and the extraction procedure. In certain preferred embodiments, the extraction solvent is water and the ratio of extraction solvent to biomass, on the basis of liters of extraction solvent to kilogram of bone-dry biomass, is from about 1:5 to about 1:100, such as from about 1:5 to about 1:50 and more preferably from about 1:5 to about 1:20.

The pH of the extraction solvent can be between about pH 5.0 and 8.0, such as, for example, between about pH 6.0 and about pH 8.0, between about pH 6.5 and about pH 7.5. In a particular embodiment, the extraction solvent is water having a pH between about pH 6.5 and about pH 7.5. In those embodiments where extraction includes imbibition with a crude juice, the imbibition fluid may have a pH from about 4.0 to about 5.0.

The extraction may be carried out at temperatures between about 25 and about 90° C., such as, for example, between about 30 and about 80° C., between about 35 and about 75° C., between about 40 and about 70° C., between about 45 and about 65° C. or between about 50 and about 60° C.

In embodiments where the extraction process is a batch extraction process, the duration of extraction may range from about 0.25 to about 24 hours, such as, for example, from about 0.5 to about 2 hours, from about 1 to about 8 hours, or from about 1 to about 6 hours.

In embodiments where the extraction process is a continuous process, the duration of extraction may range from about 0.25 to about 5 hours, such as, for example, from about 0.5 to about 3 hours.

After extraction the water insoluble biomass material may be separated from the water soluble solids by filtration to provide a filtrate containing inorganic salts, saccharides, polysaccharides, organic acids and saponins (referred to herein as the “first filtrate”). Separation can be achieved by any suitable means including, but not limited to, gravity filtration, a plate-and-frame filter press, cross flow filters, screen filters, Nutsche filters, belt filters, ceramic filters, membrane filters, microfilters, nanofilters, ultrafilters or centrifugation. Optionally various filtration aids such as diatomaceous earth, bentonite, zeolite, etc., may also be used in this process.

After separation, the pH of the first filtrate may be adjusted to remove additional impurities. In one embodiment, the pH of the first filtrate can be adjusted to between about 8.5 and about 10.0 by treatment with a base, such as, for example, calcium oxide or hydroxide (about 1.0% from the volume of filtrate) with slow agitation.

In a particularly preferred embodiment processing biomass according to the present invention removes at least about 25% of the water soluble solids from the biomass, more preferably at least about 50%, still more preferably at least about 75%, such as from about 25 to about 98%, such as from about 50 to about 90%, such as from about 75 to about 90%.

The amount of water soluble solids recovered from biomass may vary depending on the extraction efficiency, however, in certain instances from about 95 to about 350 grams of water soluble solids may be extracted per kilogram of bone dry biomass, such as from about 120 to about 315 grams per kilogram, such as from about 150 to about 300 grams per kilogram. Of the extracted water soluble solids, the total saponins may comprise from about 10 to about 30 wt %, based upon the bone dry weight of the water soluble solids. In this manner the amount of total saponins that may be extracted from biomass may range from about 10 to about 100 grams per bone dry kilogram of biomass, such as from about 20 to about 80 grams, such as from about 25 to about 75 grams. In certain instances, the amounts of materials (on bone dry grams per kilogram of bone dry biomass) removed from the biomass during the extraction process may range as set forth in Table 1, below.

TABLE 1

| | Amount |
|---|---|
| | (g/kg of bone dry biomass) |
| Total Extracted Solids | 100-400 |
| Total Water Insoluble Solids | 5-50 |
| Total Water Soluble Solids | 95-350 |
| Total Saponins | 10-100 |

In a particularly preferred embodiment milling of the biomass is carried out with the addition of an aqueous solvent, such as water, having a pH ranging from about 5 to about 9, such as from about 6 to about 7 to about 8. The water soluble solids are generally recovered from the milling process as a crude extract and may be subjected to further processing to recover specific compounds, such as saccharides, polysaccharides, organic acids and saponins.

The suspended solids, also referred to herein as the water insoluble fraction, may be removed from the crude extract by well-known processes including, for example, clarification, filtration, centrifugation, or a combination thereof. The amount of water insoluble solids in the extract (on bone dry grams per kilogram of bone dry biomass) may range from about 1.0 to about 30 grams and may comprise hydrophobic substances such as waxes and the like.

After removal of suspended solids, the clarified juice may be used directly, concentrated, or subjected to further processing to isolate one or more water soluble solids such as saccharides, polysaccharides, organic acids, saponins and sapogenins. In other instances, the clarified juice may be further purified to remove saccharides, polysaccharides, and organic acids to yield composition comprising saponins.

In particular instances saponins may be extracted and recovered from non-woody plants of the genus Hesperaloe according to the present invention. As used here, the term saponin generally refers to a compound consisting of a triterpenoid of oleanane structure and one or more glycosides, the glycosides being bound to the triterpenoid at the 3 position and/or at the 28 position. The term glycoside is intended to mean all sugars including glucose found naturally in non-woody plants of the genus Hesperaloe including arabinose, glucose, galactose, xylose, and glucuronic acid.

Saponins may obtained by sequentially extracting the biomass from non-woody plants of the genus Hesperaloe with water and then further treating the water soluble fraction with a water-immiscible polar solvent to form a polar solvent-saponin mixture. Suitable water-immiscible polar solvents include, for example, alcohols having from 4 to 6 carbon atoms, such as butyl, amyl, hexyl and cyclohexyl alcohols. The polar solvent may be removed from the saponin-containing mixture to produce a saponin-containing product.

The juice resulting from the foregoing extraction process may be subjected to further extraction to obtain saponin in the form of a crude saponin extract or its substantially purified form comprising saponins at a concentration from about 30 to about 90% in weight. The extraction method may comprise mixing juice extracted from non-woody plants of the genus Hesperaloe with a water-immiscible polar solvent. Suitable water-immiscible polar solvents include, for example, alcohols having from 4 to 6 carbon atoms, such as butyl, amyl, hexyl and cyclohexyl alcohols. Extraction of the juice with a water-immiscible polar solvent generally removes impurities such as proteins, carbohydrates, and organic acids, which remain in the aqueous phase, the saponin being transferred to the solvent phase.

The solvent phase containing the saponin may be subjected to further treatment to separate the saponin from the alcohol phase. This can be accomplished in various ways including, for example, by cooling, by dehydrating the solvent extract, or by adding an organic solvent which is miscible with the alcohol solvent but in which the saponin is insoluble. Suitable precipitating solvents include, for example, diethyl ether, petroleum ether, acetone, and chloroform.

In a particularly preferred embodiments, the saponin is separated from the alcohol by flash evaporation. Flash evaporation is a technique known in preparative chemistry for the rapid removal of a volatile component from a liquid mixture. The volatile liquid is removed from solution by rapid conversion to a vapor phase by creating a thin film of the solution over a large surface area under reduced pressure often accompanied by an increase of temperature of the solution above ambient but less than the boiling point of the solution at atmospheric pressure. The actual thickness of the film and the area over which it is applied is chosen to provide optimum evaporation and ease of use, but evaporation may be substantially instantaneous (hence the name “flash” evaporation). Flash evaporation avoids the prolonged use of high temperatures that may degrade the intended product and has the ability to remove almost all of the alcohol component (which makes the remaining solution suitable for the preferred practice of spray drying employed in the next step. The alcohol may be recovered from this step and re-used in the extraction process.

The saponin content of the alcohol extract can be further increased by passage over an ultrafiltration membrane without significant alteration to or loss of the saponin composition. This concentrated saponin fraction where the saponin content is in the range of 85-90%, can then be further purified in a liquid state or reduced to a dry state. Individual saponins may be recovered by a combination of reversed-phase solid phase extraction and preparative reversed-phase HPLC. Alternatively, the alcohol extract containing saponins can be fractionated directly by a combination of reversed-phase solid phase extraction and preparative reversed-phase HPLC.

In still other embodiments saponins may be purified from juice prepared according to the present invention comprising the steps of mixing the juice with a salt and a solvent to form a first solution. The solvent may comprise one or more solvents selected from acetic acid, acetone, acetonitrile, benzene, 1-butanol, 2-butanol, 2-butanone, t-butyl alcohol, carbon tetrachloride, chlorobenzene, chloroform, cyclohexane, 1,2-dichloroethane, diethylene glycol, diethyl ether, diglyme, 1,2-dimethoxyethane, dimethylformamide, dimethylsulfoxide, 1,4-dioxane, ethanol, ethyl acetate, ethylene glycol, glycerin, heptane, hexamethylphosphoramide, hexamethylphosphorous triamide, hexane, methanol, methyl-t-butyl ether, methylene chloride, N-methyl-2-pyrrolidinone, pentane, perchloroethylene, petroleum ether, 1-propanol, 2-propanol, pyridine, tetrahydrofuran, toluene, triethylamine, trifluorotoluene, water, xylene, or any combination of the forgoing. In some embodiments the solvent is water. The salt may be selected from an alkali metal salt, an alkaline earth salt, a transition metal salt, an ammonium salt, or combinations of the forgoing. In certain preferred embodiment the salt added to the plant extract to form the solution is an alkaline earth metal salt. In particularly preferred embodiments the salt is calcium chloride (CaCl₂)), magnesium chloride (MgCl₂), or a mixture thereof.

The pH of the first solution is generally adjusted to a pH from about 6.0 to about 9.0, such as from about 6.0 to about 8.0, such as from about 6.0 to about 7.0. At least one phosphate may then be added to the first solution to form an ion-polysaccharides complex precipitate. Useful phosphates include, for example, sodium hydrogen phosphate (Na₂HPO₄), sodium dihydrogen phosphate (NaH₂PO₄), sodium phosphate (Na₃PO₄), or sodium hydrogen bisphosphate (Na₂H₂PO₇).

The precipitated ion-polysaccharides complex may be removed by filtration to yield a second solution, which may be further clarified to produce an extract of purified saponins. Optionally, the extract can be concentrated by any filtration technique known in the prior art. Preferably, the concentration of the extract of purified saponins is carried out by nanofiltration, ultrafiltration and diafiltration, or any combination of these techniques. In some embodiments, the saponin extract is substantially free of proteins. In some embodiments, the saponin extract is substantially free of polysaccharides. In some embodiments, the saponin extract is substantially free of phenolic compounds.

In other instances, the crude or partially purified saponin may be acidified to produce sapogenins. First a solution of saponins in an alcohol is prepared as described herein and then a strong acid, preferably 1-3.5 N, is added to the solution to hydrolyze the saponins to form corresponding sapogenins. The sapogenins may be further processed by precipitation, recovering the precipitate, and decolorizing the precipitate by forming a slurry of the precipitate with a solution of an aqueous base to form a decolorized sapogenin product.

In a particularly preferred embodiment, sapogenins can be obtained by acid hydrolysis of a solution of saponins in an alcohol, for example using 450 mL concentrated HCl per 3 L of the alcohol extract under reflux. The hydrolysate is allowed to cool resulting in the formation of a precipitate which is recovered by filtration. The precipitate is slurried in water and the resulting slurry is adjusted preferably to pH 10 with a base. The sapogenins precipitate from the basic solution as off-white crystals and are recovered by filtration. The resulting crystalline precipitate may be washed with dilute acid and distilled water until the effluent is clear. The precipitate containing the sapogenins may then be air-dried and can be further refined by recrystallization.

The individual sapogenins may be recovered from this mixture, e.g. by preparative HPLC using reversed-phase adsorbents. The purification can also be achieved on a large scale by selective desorption from a reversed-phase solid-phase extraction cartridge eluted with a step gradient of aqueous methanol. Preparative HPLC and systems such as simulated moving bed chromatography are frequently in commercial use for recovery of high value solutes from solutions. The sapogenins may be further purified by recrystallization from hot 95% alcohol.

The saponin content of the various fractions during extraction may be monitored, for example, by HPLC analysis of a filtered 50% (v/v) ethanol or methanol extraction by chromatography on C-8 or C-18 RP columns eluted with a 0.05% Trifluoroacetic acid (v/v) (TFA) in water:methanol gradient, or a 0.05% TFA in water:acetonitrile gradient. Saponins in the samples may be detected by Evaporative Light Scattering Detection (ELSD) using, for example, Model PL-EMD 960 from Polymer Laboratories. Acetic acid (1%) can be used in place of TFA and chromatographic separation can be achieved by isocratic elution. The sapogenin content of extracts and samples derived by hydrolysis can also be determined using the same chromatographic procedure.

The total saponin content in Hesperaloe, on the basis of grams of total saponin per kilogram of bone dry biomass, may range from about 5.0 to about 150 g/kg, such as from about 50 to about 120 g/kg, such as from about 80 to about 120 g/kg. In other instances, water soluble solids prepared from Hesperaloe biomass according to the present invention may comprise at least about 5 wt % of total saponins, such as from about 5 to about 15 wt %, such as from about 8 to about 12 wt %, based upon the dry weight of the water soluble solids. The saponins may be provided as part of a crude juice, as part of a dried water soluble solids compositions, as a partially purified compositions or as a substantially pure composition comprising a mixture of saponins.

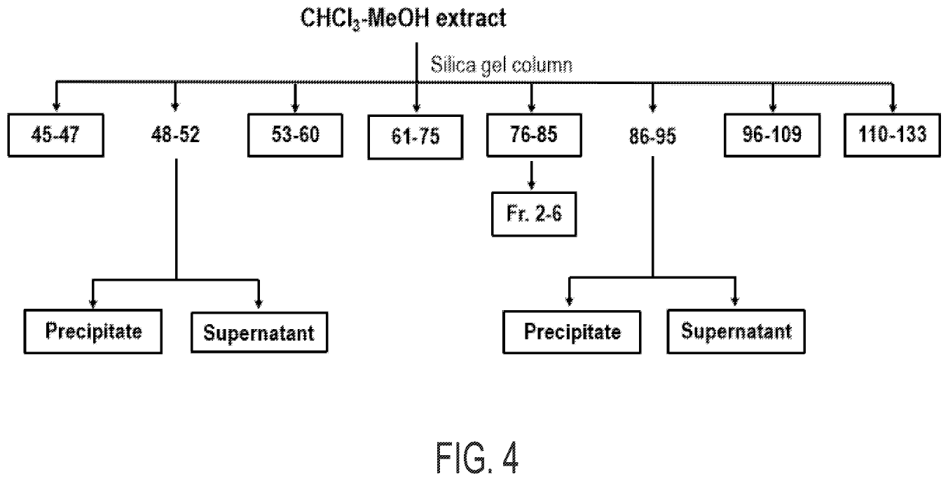

In certain embodiments saponins extracted from Hesperaloe biomass may have at least one of the following aglycones or genins: kammogenin, manogenin, gentrogenin, hecogenin, tigogenin, sarsapogenin, chlorogenin and gitogenin or their corresponding isomer or oxidized or reduced forms with at least one of the following glycosidic moieties (in the form of acid or salt): glucose, xylose, rhamnose, arabinose, or galactose. In other embodiments the saponins may comprise agamenoside, agaveside, agavoside, magueyside, agavasaponi, cantalasaponin, sisalsaponin, gabrittonoside, dongnoside or amolonin, or other steroidal saponins. In certain instances, the saponins may comprise 25(27)-dehydrofucreastatin (FIG. 4A), 5(6),25(27)-disdehydroyuccaloiside C (FIG. 4B), 5(6)-disdehydroyuccaloiside C (FIG. 4C), furcreastatin and yuccaloiside C.

Saponins prepared from Hesperaloe biomass are useful for treating a wide variety of fungal infections in plants, humans, and animals. The utility saponins prepared from Hesperaloe biomass in the treatment of infection caused by Candida albicans has been demonstrated in the Examples, below. saponins prepared from Hesperaloe biomass, particularly one or more saponins selected from 25(27)-dehydrofucreastatin (FIG. 4A), 5(6), 25(27)-disdehydroyuccaloiside C (FIG. 4B), 5(6)-disdehydroyuccaloiside C (FIG. 4C), furcreastatin and yuccaloiside C, would be useful for the treatment of infections caused by other fungal species, including, but not limited to Blastomyces, such as B. dermatitidis; Blastoschizomyces, such as B. capitatus, other species of Candida, such as C. glabrata, C. krusei, C. pseudotropicalis, and C. tropicalis; Cladosporium, such as C. carrionii; Coccidioides, such as C. immitis; Cryptococcus, such as C. neoformans; Geotrichum, such as G. clavatum; Histoplasma, such as H. capsulatum; Paracoccidioides, such as P. brasiliensis; Penicillium, such as P. marneffei; Scedosporium, such as S. apiosperum; Sporothrix, such as S. schenckii; and Trichosporon, such as T. beigelii.

The fungicidal saponin compositions of the present invention can be applied or administered by any one of a number of well-known methods and may vary depending on the locus of the fungal infection or potential fungal infection. For agronomic applications, fungicidal saponin compositions of the present invention may be applied to surfaces of plant foliage, flowers, seeds, fruits and vegetables, roots, tubers, and even the soil in the vicinity of seeds, plants, and the like. The fungicidal saponin compositions of the present invention may also be administered to human and non-human animals through various routes, including oral, nasal, rectal, parenteral, implant, topical, and the like.

In certain embodiments the fungicidal saponin compositions may delivered in a suitable solid or liquid carrier or vehicle that is compatible with the plant, human or non-human animal being treated. For example, composition of the present invention may be formulated as an aqueous spray or dip, wettable powder, drench, dust, granule, pellet, or the like, when applying the compositions to plants. In other embodiments the compositions may be formulated for topical application to humans and non-human animals by mixing the saponins with a liquid carrier such as deionized water, physiological saline, 5% (w/v) dextrose solution, vegetable oil, alcohol, propylene glycol or other liquid vehicle. Where the composition comprises a liquid carrier the concentration of saponins may range from about 2 to about 200 mg/ml, such as from about 5 to about 200 mg/ml, such as from about 10 to about 150 mg/ml, such as from about 15 to about 100 mg/ml.

In still other embodiments a composition for topical application may be prepared by incorporating one or more saponins into lotions, ointments, creams, eye, ear and nose drops, shampoos, body powders, pessaries, wound dressings, inhalers, sanitary devices, skin patches, sprays or aerosols.

In still other embodiments saponins may be safely administered orally or parenterally according to a publicly known method as a pharmaceutical composition mixed with a pharmacologically acceptable carrier, for example, as a tablet (including sugar-coated tablet and film-coated tablet), powder, granule, capsule, (including soft capsule), liquid, injection, suppository or controlled release tablet.

As the pharmacologically acceptable carrier which may be used for the preparation of pharmaceutical compositions according to the present invention, various organic or inorganic carrier substances commonly used as a material for pharmaceutical preparations can be mentioned including, for example, excipients, lubricants, binders, disintegrators for solid pharmaceutical preparations; and solvents, solubilizing agents, suspending agents, isotonizing agents, buffering agents for liquid pharmaceutical preparations. In addition, additives such as ordinary preservatives, antioxidants, colorants, sweeteners, absorbents, and wetting agents can be added as necessary.

As the excipient, for example, lactose, white sugar, D-mannitol, starch, cornstarch, crystals cellulose and light anhydrous silicic acid can be mentioned.

As the lubricant, for example, stearic acid magnesium, stearic acid calcium, talc and colloidal silica can be mentioned.

As the binder, for example, crystals cellulose, white sugar, D-mannitol, dextrin, hydroxypropylcellulose, hydroxypropylmethylcellulose, polyvinylpyrrolidone, starch, sucrose, gelatin, methylcellulose and carboxymethylcellulose sodium can be mentioned.

As the disintegrator, for example, starch, carboxymethylcellulose, carboxymethylcellulose calcium, croscarmelose sodium, sodium carboxymethyl starch and L-hydroxypropylcellulose can be mentioned.

As the solubilizing agent, for example, polyethylene glycol, propylene glycol, D-mannitol, benzyl benzoate, ethanol, trisamino methane, cholesterol, triethanolamine, sodium carbonate and citric acid sodium can be mentioned.

As the suspending agent, for example, a surfactant such as stearyltriethanolamine, sodium lauryl sulfate, lauryl aminopropionic acid, lecithin, benzalkonium chloride, benzethonium chloride and glyceryl mono stearate; and hydrophilic macromolecules, for example, polyvinyl alcohol, polyvinylpyrrolidone, carboxymethylcellulose sodium, methylcellulose, hydroxymethylcellulose, hydroxyethyl cellulose and hydroxypropylcellulose can be mentioned.

As the isotonizing agent, for example, glucose, D-sorbitol, sodium chloride, glycerol and D-mannitol can be mentioned.

In a particularly preferred embodiment one or more saponins derived from Hesperaloe according to the present invention are formulated for topical use, and any dosage form used for topical pharmaceutical compositions can be employed without any particular limitations, including lotion, liniment, poultice, ointment, patch, spirit, suspension agent, emulsion, transdermally delivered pharmaceutical preparation, liquid, cream, aerosol and nail lacquer agent. Particularly preferred are nail lacquer agent, lotion, liquid, cream, patch, and the like.

The topical anti-fungal compositions of the present invention can contain any components commonly used for pharmaceutical compositions within the scope not impairing the effect of the present invention. Such components include, for example, hydrocarbons such as vaseline and microcrystalline wax; esters such as jojoba oil, spermaceti wax, triacetin, triethyl citrate and butyl acetate; triglycerides such as beef tallow and olive oil; higher alcohols such as cetanol and oleyl alcohol; fatty acids such as stearic acid and oleic acid; alcohols such as ethanol and isopropanol; polyalcohols such as glycerol and 1,3-butane diol; water, nonionic surfactants, anionic surfactants, cationic surfactants, amphoteric surfactants, polyvinylpyrrolidone, thickeners such as Carbopol, preservatives, ultraviolet absorbers, antioxidants, pigments and powders.

Generally anti-fungal compositions of the present invention comprise a quantity of Hesperaloe derived saponins sufficient to obtain a reduction in the level of disease, as measured by fungal growth or the symptoms associated with fungal growth, relative to that occurring in an untreated control under suitable conditions of treatment as described herein. In certain embodiments the anti-fungal compositions are effective against C. albicans at levels less than about 20 μg/mL, such as less than about 15 μg/mL, such as from about 5 to about 20 μg/mL. The particular dose regimen, however, will be dependent upon a plurality of factors, such as the species, size, sex, and age of the individual being treated, the target fungal species, the severity of infection, the mode of administration, etc. Upon taking these factors into account, actual dose level and regimen could be readily determined by the person of ordinary skill in the art.

The amount of saponin in a given pharmaceutical preparation of the present invention may range from about 0.01 to 100 wt % based on the total pharmaceutical preparation, such as from about 0.5 to about 50 wt %, such as from about 1.0 to about 10 wt %.

### Test Methods

### Water Soluble Solids

Total biomass water soluble solids may be determined using an Accelerated Solvent Extraction system (ASE) such as a Dionex™ ASE™ 350 (Thermo Fisher Scientific, Waltham, MA). Approximately 10 grams of harvested biomass is dried to a constant weight in an oven, typically 4 hours at 125° C. After drying 1.5-2.0 grams of the bone dry biomass is accurately weighed and the weight (W_{b}) recorded to the nearest 0.001 gram. Using water as the solvent, biomass is extracted using the conditions set forth in the table below. The ratio of biomass to solvent is generally 21:1 and five consecutive water extraction cycles are performed. At the end of each extraction cycle, the liquid phase is collected, dried under vacuum at approximately 40° C. and the weight of the dried material (W₁) is recorded to the nearest 0.001 g. The total weight of water soluble solids (Wₑ) is calculated by summing the weight of solids recovered from each extraction cycle (W₁). Total water soluble solids as a percentage of bone dry biomass is then determined using the following equation: Water Soluble Solids (wt %)=Wₑ/W_{b}*100.

| | |
|---|---|
| Pressure (psi) | 1500 |
| Temperature (° C.) | 40 |
| Static Time (min.) | 10 |
| Cycles (no.) | 5 |

The total water soluble solids in biomass extract may be determined by withdrawing an appropriate aliquot, typically about 10-50 ml, transferring to clean, dry, centrifuge tube. The tube is centrifuged at 7000 rpm for 20 minutes. The weight of extract (W₁) is calculated. An aliquot of the supernatant is then transferred to clean, pre-weighed beaker (D₀), and weighed. The beaker and sample are then weighed to the nearest 0.001 g and the weight (D₂) recorded. The beaker containing the sample is then placed at 140° C. in a hot air oven for overnight drying. The beaker is removed from the oven and desiccated to cool to room temperature then weighed to the nearest 0.001 gram (D₁). The weight percentage of soluble solids, based upon the weight of the extract, is determined using the formula below:

$$Water Soluble Solids ( wt \% ) = \frac{( {D}_{1} - {D}_{0} ) \times 100}{( {D}_{2} - {D}_{0} )}$$

D₁=mass of empty beaker+dried soluble solids, D₀=mass of empty beaker, D₂=mass of biomass extract and empty beaker.

### Total Saponins

Total saponins were measured generally as described in Makkar, Harinder P. S., Sidhuraju, P., Becker, Klaus (2007) Plant Secondary Metabolites, chapter 17, pp 93-100. A standard saponin solution was prepared by weighing 10 mg of diosgenin (MilliporeSigma >93%), dissolving in 16 mL of methanol and adding 4 mL of distilled water. The solution was mixed thoroughly to yield a 0.5 mg/ml diosgenin solution in 80% methanol solvent. The standard was used to produce a calibration curve by transferring various amounts of the standard (0, 10, 20, 40, 60, 80, and 100 μL) into 13-mm glass test tubes. A solution of 80% aqueous methanol was added to a total volume of 100 μL.

Prior to testing samples of biomass extract were adjusted to about 0.5 wt % total solids by dilution with water to ensure absorbency result fell along the saponin standard calibration curve range. Samples of diluted extract (20-μL) were pipetted into 13-mm glass test tubes and the volume was brought up to 100 μL with 80 μL methanol. Each sample was tested in triplicate.

To each sample 100 μL of vanillin reagent (prepared by dissolving 800 mg of vanillin in 10 ml of 99.5% ethanol (analytical grade)) and then 1.0 ml of 72% (v/v) sulfuric acid (72% (v/v) sulfuric acid prepared by adding 72 mL of sulfuric acid (analytical grade, 95%, w/w) to 28 mL of distilled water) were added. Solutions were mixed well and heated at 60° C. for 10 minutes. Samples were then cooled in an ice bath and 1 mL of solution was transferred into respective cuvette and absorbance at 544 nm was read. The total mass of saponins in the sample may be calculated based upon the standard absorbency curve as follows:

$$Saponin ( μ g ) = [ Slope ] \times Measured Absorbency - [ Intercept ]$$

### EXAMPLE

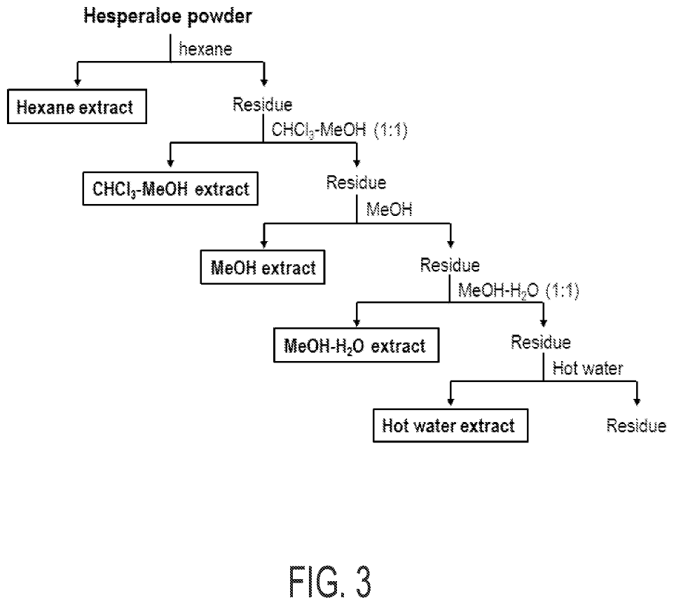

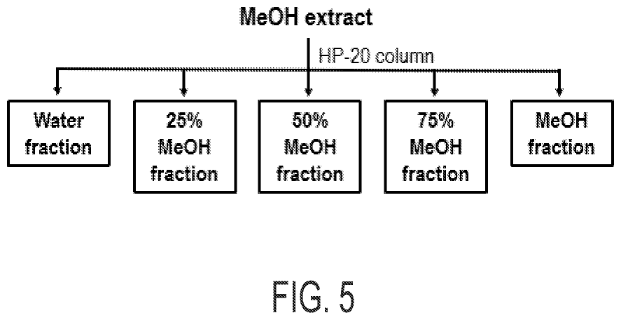

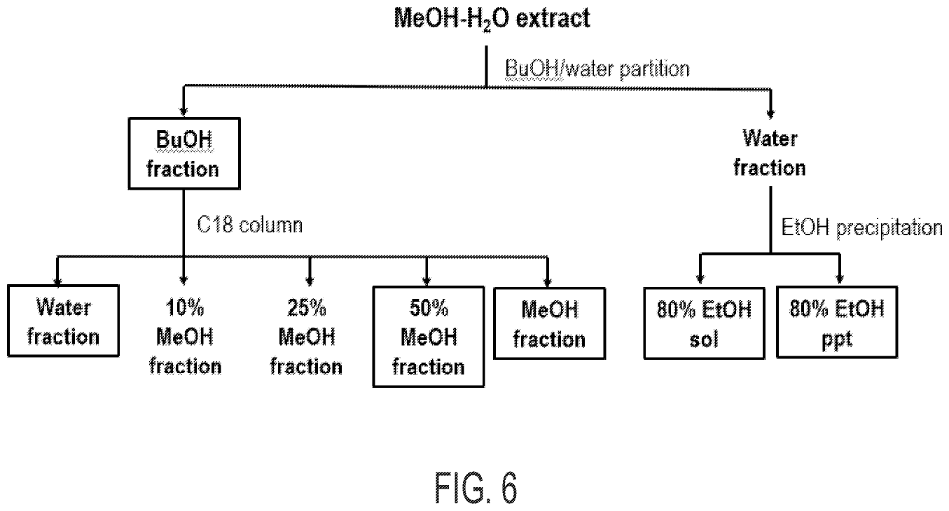

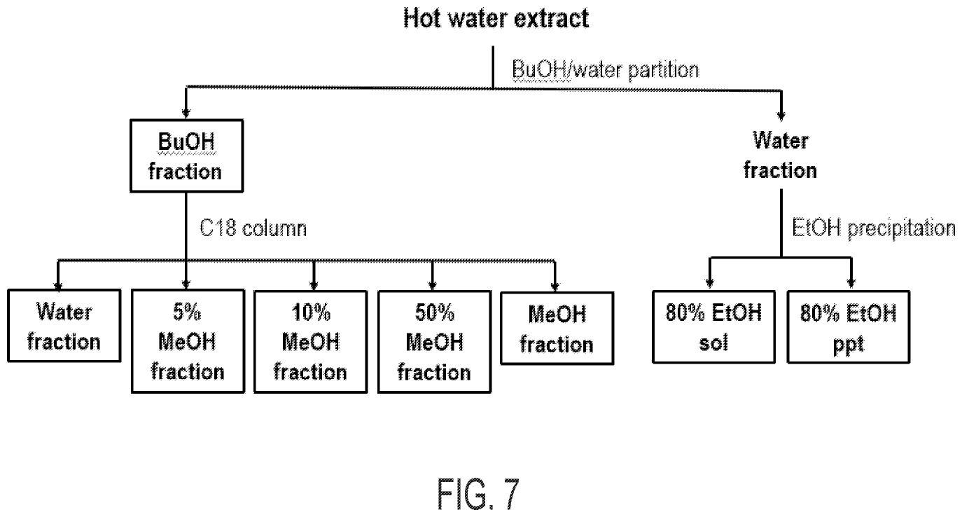

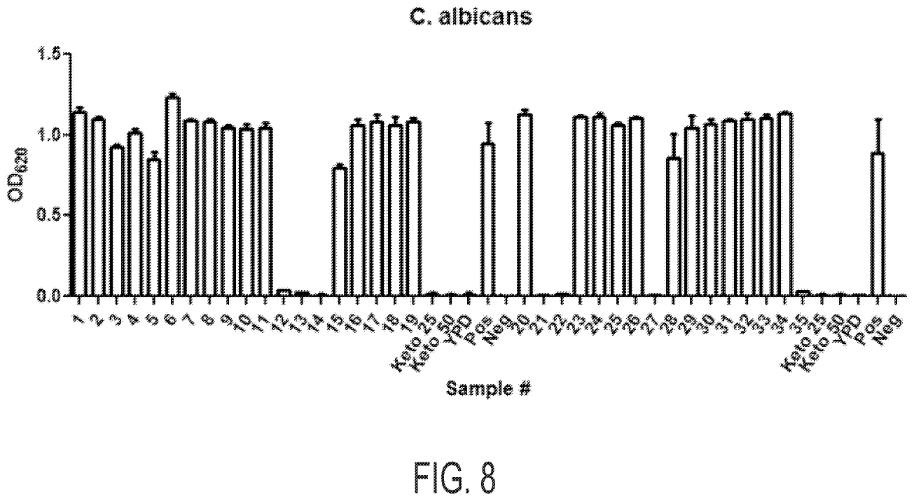

Five-hundred (500) grams of dry, coarsely ground Hesperaloe funifera was sequentially extracted by soaking (15 h) biomass with occasional stirring using Hexane (H), 1:1 Chloroform/Methanol (CM), Methanol (M), Methanol/Water (MW) and Hot Water (HW) to yield a crude extract. The extraction scheme is illustrated in FIGS. 3-7 and further described below. Extraction conditions and yields are provided in Table 2, below. All extracts were reduced in volume using rotary evaporator and freeze dried.

TABLE 2

| | Amount | Conditions | Yield | Yield |
|---|---|---|---|---|
| Solvent | (L) | (° C.) | (g) | (%) |
| Hexanes | 20 | RT | 3.18 | 0.64 |
| CHCl₃—MeOH (1:1) | 20 | 30-40 | 36.76 | 7.35 |
| MeOH | 21 | 30-40 | 25.08 | 5.02 |
| MeOH—H₂O (1:1) | 20 | 78-80 | 32.03 | 6.41 |
| H₂O | 20 | 78-80 | 24.27 | 4.85 |

The extracts were subjected to partitioning and purification using high performance liquid chromatography/mass spectrometry (HPLC/MS), which yielded 30 fractions, summarized in Table 3.

TABLE 3

| Sample # | Sample ID | Sample Description |
|---|---|---|
| 1 | H | Hexane extract |
| 2 | CM | CHCl₂—MeOH (1:1) extract |
| 3 | M | MeOH extract |
| 4 | MW | MeOH—H₂O (1:1) extract |
| 5 | HW | Hot water extract |
| 6 | CM F45-47 | Fraction 45-47 from CHCl₂—MeOH (1:1) extract |
| 7 | CM F48-52 ppt | Fraction 48-52 precipitate from CHCl₂—MeOH (1:1) extract |
| 8 | CM F48-52 sup | Fraction 48-52 supernatant from CHCl₂—MeOH (1:1) extract |
| 9 | CM F53-60 | Fraction 53-60 from CHCl₂—MeOH (1:1) extract |
| 10 | CM F61-75 | Fraction 61-75 from CHCl₂—MeOH (1:1) extract |
| 11 | CM F76-85 | Fraction 76-85 from CHCl₂—MeOH (1:1) extract |
| 12 | CM F86-95 ppt | Fraction 86-95 precipitate from CHCl₂—MeOH (1:1) extract |
| 13 | CM F86-95 sup | Fraction 86-95 supernatant from CHCl₂—MeOH (1:1) extract |
| 14 | CM F96-109 | Fraction 96-109 from CHCl₂—MeOH (1:1) extract |
| 15 | CM F110-133 | Fraction 110-133 from CHCl₂—MeOH (1:1) extract |
| 16 | CM F76-85 F2-6 | Fraction 2-6 from Fraction 76-85 from CHCl₂—MeOH (1:1) extract |
| 17 | M-HP20-W | Water fraction from MeOH extract |
| 18 | M-HP20-25M | 25% MeOH fraction from MeOH extract |
| 19 | M-HP20-50M | 50% MeOH fraction from MeOH extract |
| 20 | M-HP20-75M | 75% MeOH fraction from MeOH extract |
| 21 | M-HP20-M | MeOH fraction from MeOH extract |
| 22 | MW-B | n-BuOH fraction from MeOH—H₂O (1:1) extract |
| 23 | MW-80E sol | 80% EtOH soluble fraction from MeOH—H₂O (1:1) extract |
| 24 | MW-80E ppt | 80% EtOH precipitate from MeOH—H₂O (1:1) extract |
| 25 | MW-B-C18-W | Water fraction from BuOH fraction from MeOH—H₂O (1:1) extract |
| 26 | MW-B-C18-50M | 50% MeOH fraction from BuOH fraction from MeOH—H₂O (1:1) extract |
| 27 | MW-B-C18-M | MeOH fraction from BuOH fraction from MeOH—H₂O (1:1) extract |
| 28 | HW-B | n-BuOH fraction from hot water extract |
| 29 | HW-80E sol | 80% EtOH soluble fraction from hot water extract |
| 30 | HW-80E ppt | 80% EtOH precipitate from hot water extract |
| 31 | HW-B-C18-W | Water fraction from BuOH fraction from hot water extract |
| 32 | HW-B-C18-5M | 5% MeOH fraction from BuOH fraction from hot water extract |
| 33 | HW-B-C18-10M | 10% MeOH fraction from BuOH fraction from hot water extract |
| 34 | HW-B-C18-50M | 50% MeOH fraction from BuOH fraction from hot water extract |
| 35 | HW-B-C18-M | MeOH fraction from BuOH fraction from hot water extract |

### Fraction of CHCl₃-MeOH (1:1) Extract

Fractionation of the CHCl₃-MeOH (1:1) extract was performed by normal phase chromatography using a CombiFlash system. Sample of 6 g was dissolved in CHCl₃, pre-absorbed on Si gel and loaded onto prepacked 330 g Si column preconditioned with CHCl₃ (solvent A). The column was eluted with a gradient of 0-100% MeOH with 0.5% H₂O (solvent B). The fractions were combined based on their TLC profile, yielding the following pooled fractions: Fr. 14-44, 45-47, 48-52, 53-60, 61-75, 76-85, 86-95, 96-109, and 110-133.

### Fraction of MeOH Extract

Fractionation of the MeOH extract was carried on a HP-20 column due to solubility issue. MeOH extract (10 g) dissolved in MeOH/H₂O was pre-absorbed on Diaion HP-20 resin (250 mL) and the solvent was removed. It was then loaded on the HP-20 column (2 L Buchner funnel containing 1 L of HP-20 resin preconditioned with dH₂O). The column was first eluted with 2 L dH₂O, then with the same volume of H₂O/MeOH in different concentrations to yield five fractions, namely M-HP20-W, -25M, -50M, -75M, and -M.

### Fraction of the MeOH—H₂O (1:1) and Hot Water Extracts

These two extracts were first fractioned by partitioning (liquid-liquid extraction) with butanol, followed by ethanol precipitation. The dry extract samples were dissolved in water and repeatedly extracted with equal volumes of n-BuOH pre-saturated with water. The n-BuOH soluble fraction was concentrated on the rotary evaporator and freeze dried (MW-B and HW-B).

TABLE 4

| | | LC-MS | |
|---|---|---|---|
| Molecular | | Retention time | Structure |
| formula | Fraction ID | (RT) | info |
| C₃₂H₅₂O₈ | CM F48-52 sup F27-28 | 2.36 | 1 sugar |
| C₃₃H₅₂O₉ | CM F48-52 sup F29-31 | 2.94 | 1 sugar |
| C₃₃H₅₆O₉ | CM F48-52 sup F27-28 | 2.50 | 1 sugar |
| C₃₉H₆₂O₁₃ | CM F61-75 F28-30 | 2.81 | 2 sugars |
| | CM F61-75 F31 | 2.84 | |
| C₃₉H₆₂O₁₃ | CM F61-75 F32-34 | 3.03 | 2 sugars |
| C₃₉H₆₂O₁₄ | CM F76-85 F27-29 | 2.52 | 2 sugars |
| C₃₉H₆₂O₁₄ | CM F61-75 F28-30 | 2.60 | 2 sugars |
| | CM F61-75 F31 | 2.62 | |
| | CM F76-85 F27-29 | 2.62 | |
| | CM F76-85 F30-31 | 2.61 | |
| C₃₉H₆₂O₁₄ | CM F61-75 F31 | 2.84 | 2 sugars |
| C₃₉H₆₄O₁₅ | CM F76-85 F21-23 | 1.85 | 2 sugars |
| C₄₅H₇₀O₁₇ | CM F61-75 F32-34 | 3.03 | 3 sugars |
| C₄₅H₇₂O₁₇ | CM F61-75 F32-34 | 3.11 | 3 sugars |
| C₄₅H₇₂O₁₇ | CM F76-85 F30-31 | 2.85 | 3 sugars |
| C₄₅H₇₂O₁₈ | CM F76-85 F30-31 | 2.73 | 3 sugars |
| C₅₁H₈₀O₂₂ | CM F76-85 F30-31 | 2.93 | 4 sugars |
| C₅₁H₈₂O₂₂ | CM F76-85 F30-31 | 3.01 | 4 sugars |
| | CM F76-85 F32-33 | 3.03 | |
| C63H₁₀₀O₃₂ | CM F86-95 ppt RS12-97-5 | 2.89 | 6 sugars |
| C₆₃H₁₀₀O₃₃ | CM F86-95 ppt RS12-97-2 | 2.57 | 6 sugars |
| C₆₃H₁₀₂O₃₂ | CM F86-95 ppt RS12-97-6 | 2.95 | 6 sugars |
| | CM F96-109 F20-24 | 2.95 | |
| C₆₃H₁₀₂O₃₃ | CM F86-95 ppt RS12-97-4 | 2.61 | 6 sugars |
| | CM F96-109 F15-19 | 2.62 | |
| | M-HP20-M F40 ppt | 2.58 | |
| | MW-B-C18-M F14 ppt | 2.59 | |
| C₆₃H₁₀₂O₃₃ | M-HP20-M F40 ppt | 2.76 | 6 sugars |
| | MW-B-C18-M F14 ppt | 2.80 | |
| C₆₃H₁₀₄O₃₂ | CM F86-95 ppt RS12-97-6 | 3.01 | 6 sugars |
| | CM F96-109 F20-24 | 3.00 | |
| | M-HP20-M F40 ppt | 2.96 | |
| | MW-B-C18-M F14 ppt | 2.99 | |
| C₆₃H₁₀₄O₃₂ | M-HP20-M F40 ppt | 3.14 | 6 sugars |

The novel saponins, 25(27)-dehydrofucreastatin (FIG. 4A), 5(6),25(27)-disdehydroyuccaloiside C (FIG. 4B), and 5(6)-disdehydroyuccaloiside C (FIG. 4C) were identified in fractions CM F86-95 ppt RS12-97-5, CM F86-95 ppt RS12-97-2 and CM F86-95 ppt RS12-97-6, respectively.

### Bioassay

Candida albicans, was purchased from ATCC. Thirty-five samples derived from KC Hesperaloe funifera, crude extracts and selected fractions (Table 1) were screened for the inhibition of growth of candida.

Minimum inhibitory concentration (MIC) testing was performed. Two-fold dilutions of sample prepared in C. albicans growth medium (Yeast Peptone Dextrose, YPD) broth, starting at 200 □g/mL. Then inoculum added and incubated at 30° C. for 18-24 hours, and OD₆₂₀ was determined. T-test was used to determine statistically significant decreases in growth (*=p>0.01). Controls included Ketoconazole (50 □g/mL, Keto50), Neg (media with DMSO only, no inoculum), Pos (media with DMSO and inoculum), and YPD (broth only). In wells with decreased growth, wells were mixed by pipetting, and 10 IL samples were plated on YPD agar to determine whether viable C. albicans were present, as an estimation of minimum fungicidal concentration (MFC).

TABLE 5

| | | Minimal Fungicidal |
|---|---|---|
| Sample | MIC mg/ml | Concentration Estimate mg/ml |
| 12 | 6.25 | 12.5 |
| 13 | 50 | 200 |
| 14 | 100 | >200 |
| 21 | 50 | >200 |
| 22 | 100 | >200 |
| 27 | 25 | 100 |
| 35 | 100 | >200 |
| Ketoconazole | 25 | |

What is claimed is:

1. An anti-fungal composition comprising a saponin having the formula: 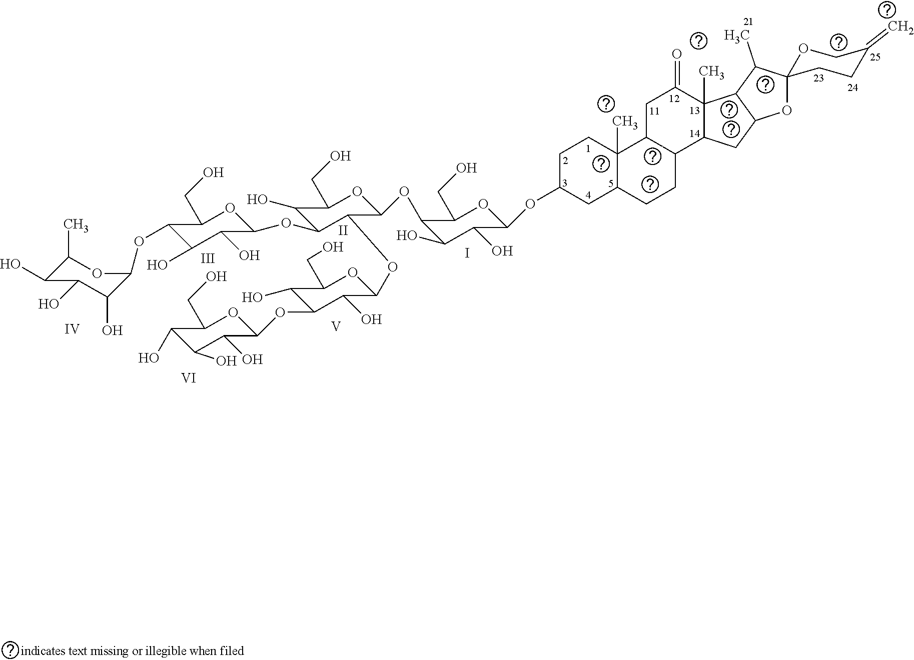
and a carrier, wherein the saponin is present in an amount effective to inhibit the growth of a fungal organism.

2. The composition of claim 1 wherein the fungal organism is of the genus Blastomyces, Candida, Cryptococcus, Epidermophyton, Microsporum, Trichophyton or Pityrosporum.

3. The composition of claim 1 wherein the fungal organism is Blastomyces dermatidis, Candida albicans, Candida guillermoundi, Cryptococcus neoformans, Microsporum canis, Microsporum audouinii, Microsporum gypseum, Trichophyton tonsuran, Trichophyton rubrum, Trichophyton mentagrophyte, Epidermophyton floccosum, Pityrosporum ovale, or a combination thereof.

4. The composition of claim 1 wherein the fungal organism is Candida albicans or Candida guillermoundi.

5. The composition of claim 1 wherein the composition further comprises a liquid carrier and the concentration of saponins ranges from about 10 to about 100 mg of saponins per milliliter of composition.

6. The composition of claim 1 wherein the saponin is extracted from a non-woody plant of the genus Hesperaloe.

7. The composition of claim 6 wherein the non-woody plant is Hesperaloe funifera, Hesperaloe nocturna, Hesperaloe parviflora, Hesperaloe chiangii, or a combination thereof.

8. The composition of claim 6 wherein the non-woody plant is Hesperaloe funifera.

9. The composition of claim 1 further comprising a saponin having the formula:

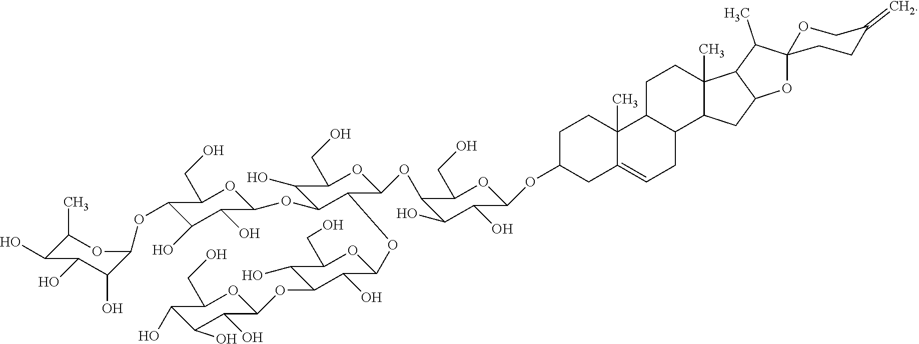 [SMILES: C=C1CCC2(OC1)OC1CC3C4CC=C5CC(OC6OC(CO)C(OC7OC(CO)C(O)C(OC8OC(CO)C(OC9OC(C)C(O)C(O)C9O)C(O)C8O)C7OC7OC(CO)C(O)C(OC8OC(CO)C(O)C(O)C8O)C7O)C(O)C6O)CCC5(C)C4CCC3(C)C1C2C]

10. The composition of claim 1 further comprising a saponin having the formula:

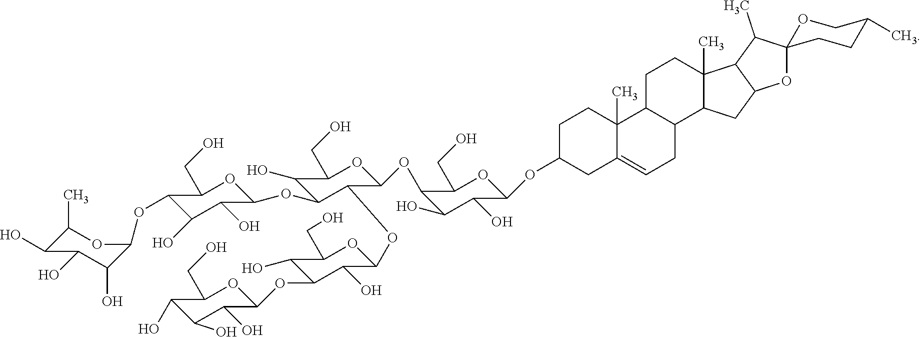 [SMILES: CC1CCC2(OC1)OC1CC3C4CC=C5CC(OC6OC(CO)C(OC7OC(CO)C(O)C(OC8OC(CO)C(OC9OC(C)C(O)C(O)C9O)C(O)C8O)C7OC7OC(CO)C(O)C(OC8OC(CO)C(O)C(O)C8O)C7O)C(O)C6O)CCC5(C)C4CCC3(C)C1C2C]

11. The composition of claim 1 further comprising furcreastatin and yuccaloiside C.

12. An anti-fungal composition comprising one or more saponins selected from the group consisting of 25(27)-dehydrofucreastatin, 5(6),25(27)-disdehydroyuccaloiside C, 5(6)-disdehydroyuccaloiside C, furcreastatin and yuccaloiside C, extracted from a non-woody plant of the genus Hesperaloe.

13. The composition of claim 12 wherein the non-woody plant is Hesperaloe funifera, Hesperaloe nocturna, Hesperaloe parviflora, Hesperaloe chiangii or a combination thereof.

14. The composition of claim 12 further comprising a pharmaceutically acceptable carrier.

15. The composition of claim 12, wherein the one or more saponins are provided in an amount to treat or prevent a fungal infection in a plant, a non-human animal, or a human.

16. The composition of claim 15, wherein the fungal infection is caused by Blastomyces dermatidis, Candida albicans, Candida guillermoundi, Cryptococcus neoformans, Microsporum canis, Microsporum audouinii, Microsporum gypseum, Trichophyton tonsuran, Trichophyton rubrum, Trichophyton mentagrophyte, Epidermophyton floccosum, Pityrosporum ovale, or a combination thereof.

17. A method for inhibiting the growth of a fungal organism in a subject comprising administering to the subject an effective amount of an anti-fungal composition comprising a saponin having the formula: 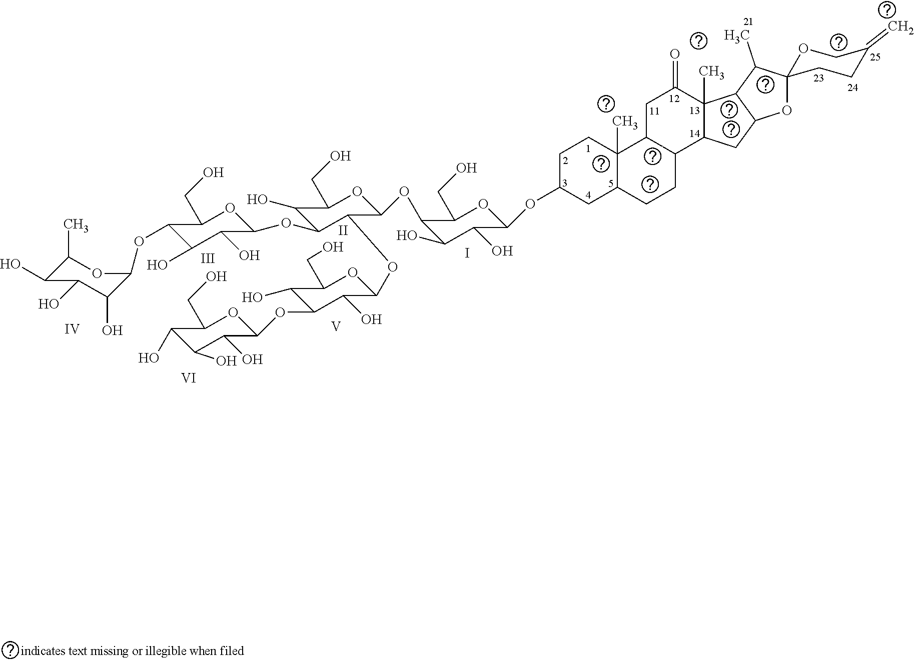
and a carrier, wherein the saponin is present in an amount effective to inhibit the growth of a fungal organism.

18. The method of claim 17, wherein the fungal organism is of the genus Blastomyces, Candida, Cryptococcus, Epidermophyton, Microsporum, Trichophyton or Pityrosporum.

19. The method of claim 17, wherein the fungal organism is Blastomyces dermatidis, Candida albicans, Candida guillermoundi, Cryptococcus neoformans, Microsporum canis, Microsporum audouinii, Microsporum gypseum, Trichophyton tonsuran, Trichophyton rubrum, Trichophyton mentagrophyte, Epidermophyton floccosum, Pityrosporum ovale, or a combination thereof.

20. The method of claim 17, wherein the fungal organism is Candida albicans or Candida guillermoundi.

21. The method of claim 17, wherein the carrier is a liquid and the concentration of saponins ranges from about 10 to about 100 mg of saponins per milliliter of composition.

22. The method of claim 17, wherein the saponin is extracted from a non-woody plant of the genus Hesperaloe.

23. The method of claim 17, wherein the non-woody plant is Hesperaloe funifera, Hesperaloe nocturna, Hesperaloe parviflora, Hesperaloe chiangi or a combination thereof

24. The method of claim 17, wherein the composition further comprises a saponin having the formula:

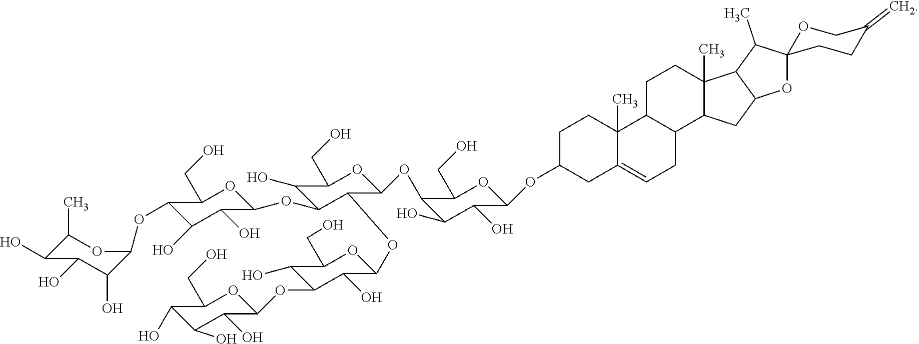 [SMILES: C=C1CCC2(OC1)OC1CC3C4CC=C5CC(OC6OC(CO)C(OC7OC(CO)C(O)C(OC8OC(CO)C(OC9OC(C)C(O)C(O)C9O)C(O)C8O)C7OC7OC(CO)C(O)C(OC8OC(CO)C(O)C(O)C8O)C7O)C(O)C6O)CCC5(C)C4CCC3(C)C1C2C]

25. The composition of claim 17 further comprising a saponin having the formula:

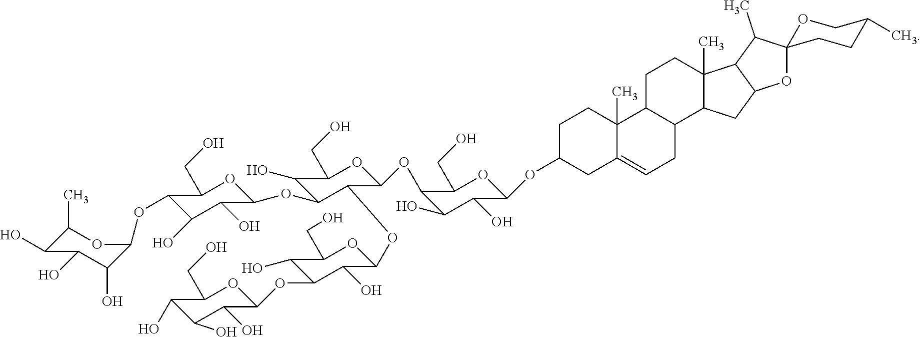 [SMILES: CC1CCC2(OC1)OC1CC3C4CC=C5CC(OC6OC(CO)C(OC7OC(CO)C(O)C(OC8OC(CO)C(OC9OC(C)C(O)C(O)C9O)C(O)C8O)C7OC7OC(CO)C(O)C(OC8OC(CO)C(O)C(O)C8O)C7O)C(O)C6O)CCC5(C)C4CCC3(C)C1C2C]

26. The composition of claim 17 further comprising furcreastatin and yuccaloiside C.
